(19) United States
(12) Patent Application Publication
(10) Pub. No.: US 2017/0095154 A1
(43) Pub. Date: Apr. 6, 2017

(54) Smart Physiological Monitoring System

(71) Applicant: Benjamin Knutsen, Sunol, CA (US)

(72) Inventors: Benjamin Knutsen, Scottsdale, AZ (US); Stephen Poole, Tewksbury, MA (US); Steven Fucci, Phoenix, AZ (US)

(21) Appl. No.: 15/233,788

(22) Filed: Aug. 10, 2016

Related U.S. Application Data
(60) Provisional application No. 62/203,263, filed on Aug. 10, 2015.

(51) Int. Cl. A61B 5/00; A61B 5/0205

(52) U.S. Cl. CPC A61B 5/0024; A61B 5/02055; A61B 5/0022; A61B 2503/04; A61B 2503/08; A61B 5/024

(57) ABSTRACT

A smart physiological monitoring system. In a first embodiment, a smart physiological monitoring system comprises a smart physiological monitoring apparatus communicably coupled with an Internet router and a mobile computing device via wireless transmission circuitry, the wireless communication circuitry of the smart physiological monitoring apparatus comprising Wi-Fi communication circuitry and Bluetooth communication circuitry. The smart physiological monitoring apparatus further comprising a sensor network, the sensor network comprising at least one physiological parameter sensor.

### CROSS REFERENCE TO RELATED APPLICATIONS

The present application claims the benefit of priority to U.S. Provisional Application Ser. No. 62/203263 titled “Smart Physiological Monitoring System” filed Aug. 10, 2015, the contents of which are hereby incorporated by reference in their entirety.

### FIELD

The present invention relates generally to monitoring systems and methods and more specifically to physiological monitoring systems and methods for smart monitoring of physiological, vital, and other parameters.

### BACKGROUND

Monitoring vital or physiological signs is crucial when caring for a child or patient. There are many separate instruments for monitoring a subject's heart rate, oxygen saturation, blood pressure, respiration rate, and body temperature.

Utilizing multiple instruments to monitor a subject under care is cumbersome, intrusive, and causes clutter in an environment where peace and order are preferred.

It is within the aforementioned context that a need for the present invention has arisen. Thus, there is a need to address one or more of the foregoing disadvantages of conventional systems and methods, and the present invention meets this need.

### SUMMARY

Various aspects of methods and systems for smart physiological monitoring can be found in exemplary embodiments of the present invention.

In a first embodiment, a smart physiological monitoring system comprises a smart physiological monitoring apparatus communicably coupled with an Internet router and a mobile computing device via wireless transmission circuitry, the wireless communication circuitry of the smart physiological monitoring apparatus comprising Wi-Fi communication circuitry and Bluetooth communication circuitry. The smart physiological monitoring apparatus further comprising a sensor network, the sensor network comprising at least one physiological parameter sensor.

With the present invention, physiological and other parameters of a person can be captured and monitored via the Internet of things, hard wire, private network or any other means of communication acting as a hub to distribute data to client connected devices. The monitored statistics are captured without physical contact with the person.

With the present invention, an infant, child, or patient can be safely monitored remotely. The present invention provides benefits not found in prior systems because the present invention captures physiological parameters, rather than just audio and visual data. Physiological parameters provide insight as to a physical condition of the person under monitoring, providing critical real time information should the physical condition be potentially harmful to the person. The present invention provides local and remote alerts based on the physical condition of the person being monitored.

With the present invention, multiple monitors are integrated into one wireless scanning device. The device eliminates intrusive and cumbersome cables that tether the body to multiple external devices. The wires often become detached from general movement, restless sleep and take up space in a crib. Removing tethers from the baby eliminates the choking hazards and discomfort of current monitors.

With the present invention, an alarm can be activated when physiological parameters reach an unsafe threshold caused by oxygen deprivation. The devices early detection system monitors for life threatening events and allows time for intervention. Early detection and more prevention time reduce unnecessary subject fatalities.

A further understanding of the nature and advantages of the present invention herein may be realized by reference to the remaining portions of the specification and the attached drawings. Further features and advantages of the present invention, as well as the structure and operation of various embodiments of the present invention, are described in detail below with respect to the accompanying drawings. In the drawings, the same reference numbers indicate identical or functionally similar elements.

### BRIEF DESCRIPTION

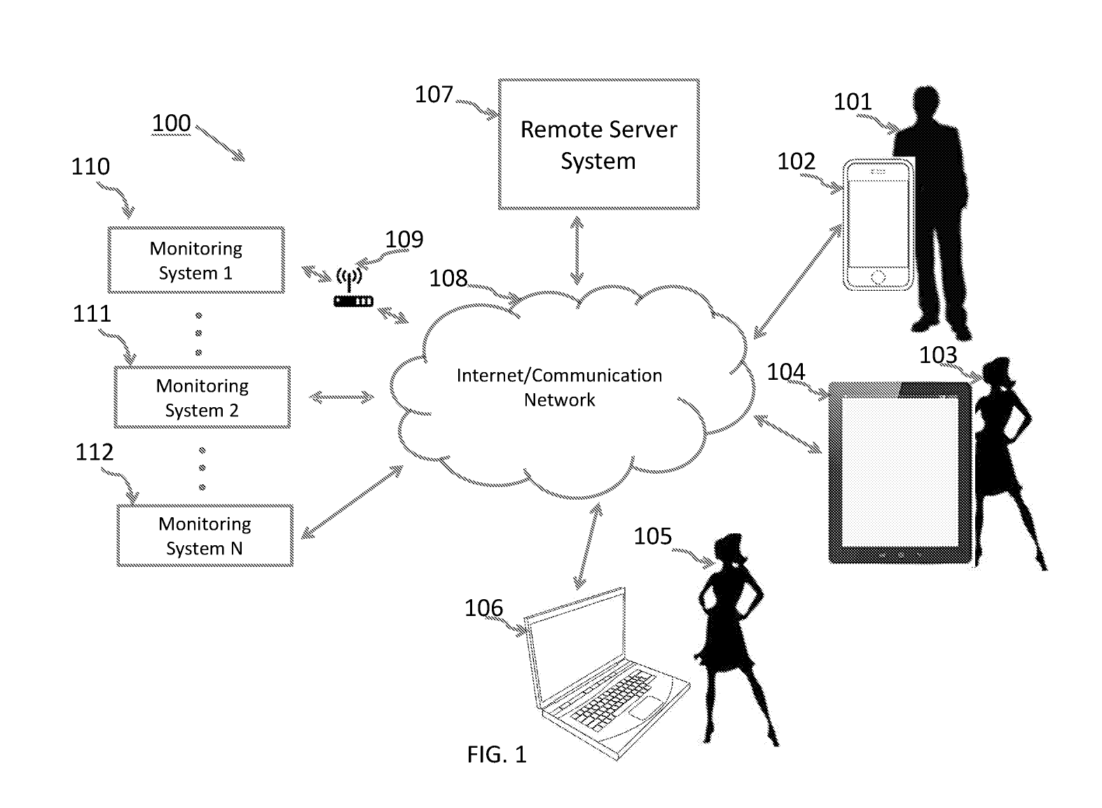

FIG. 1 illustrates a smart physiological monitoring system according to an exemplary embodiment of the present invention.

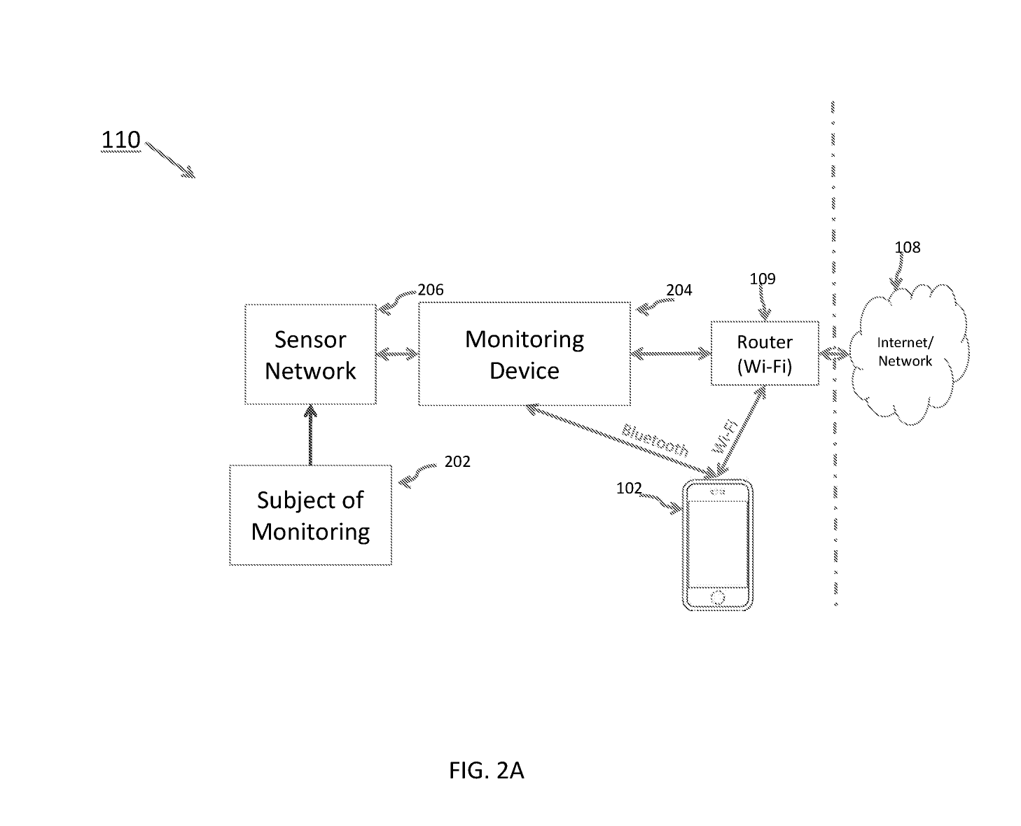

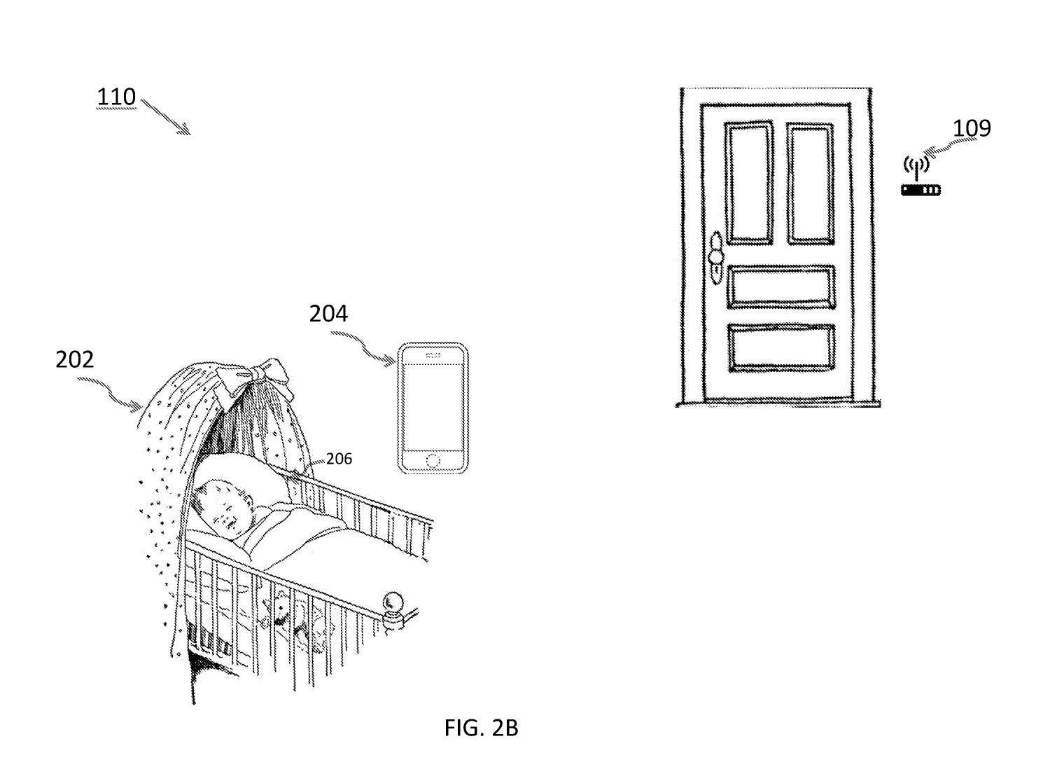

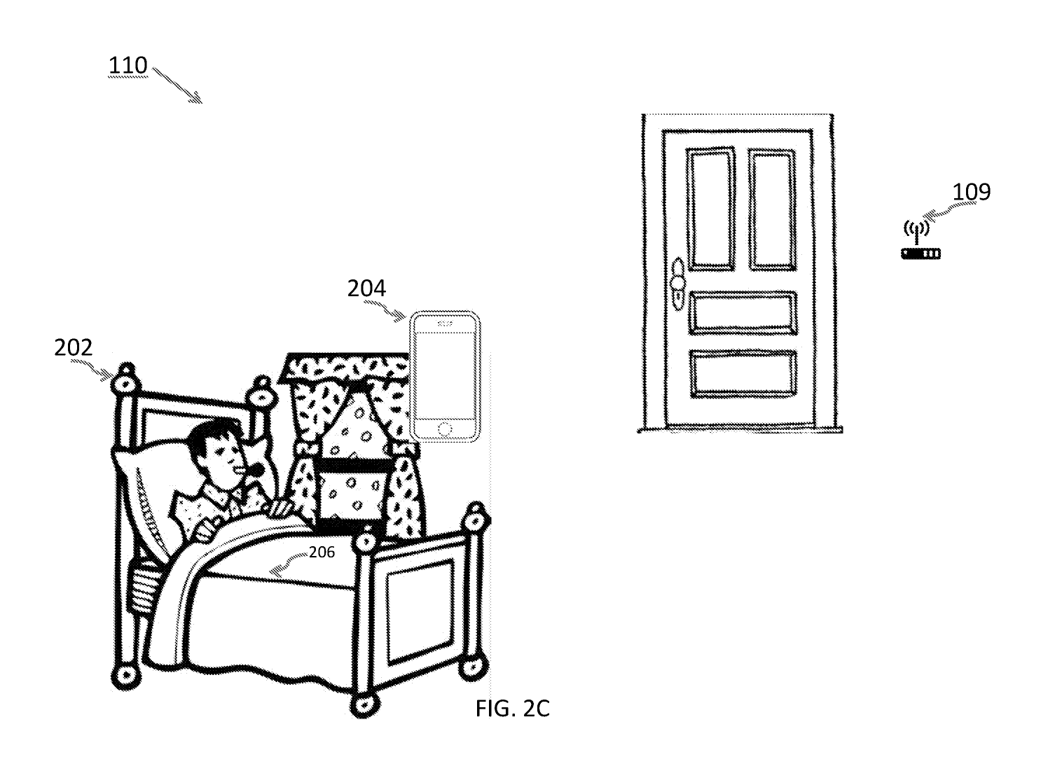

FIG. 2A illustrates smart physiological monitoring system according to an exemplary embodiment of the present invention.

FIG. 2B illustrates smart physiological monitoring system according to an exemplary embodiment of the present invention.

FIG. 2C illustrates smart physiological monitoring system according to an exemplary embodiment of the present invention.

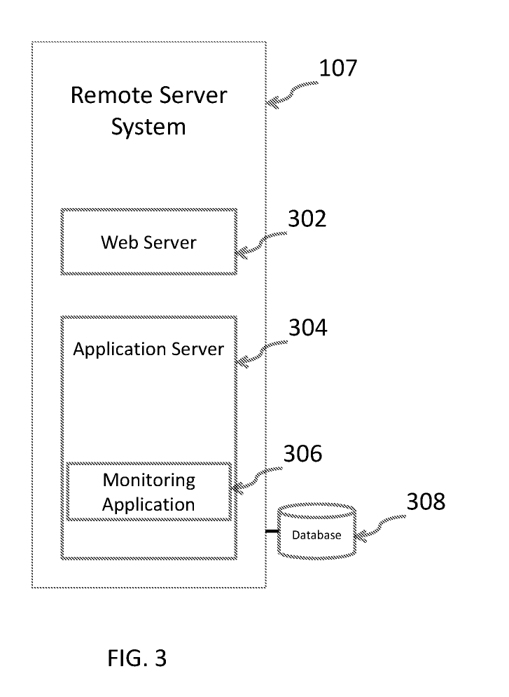

FIG. 3 illustrates remote server system according to an exemplary embodiment of the present invention.

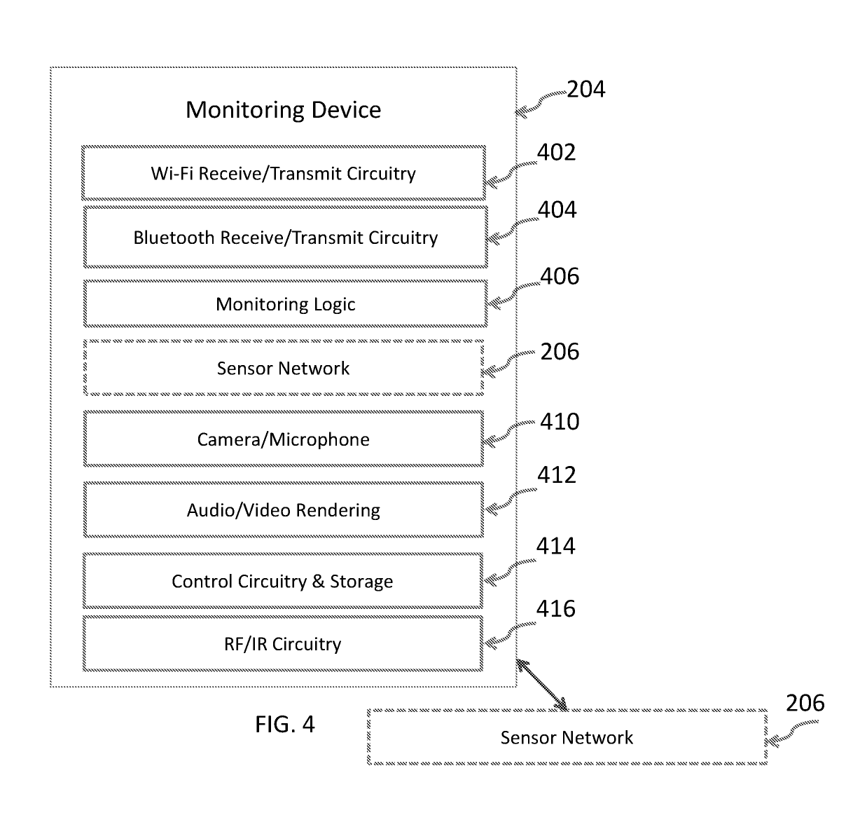

FIG. 4 illustrates monitoring device according to an exemplary embodiment of the present invention.

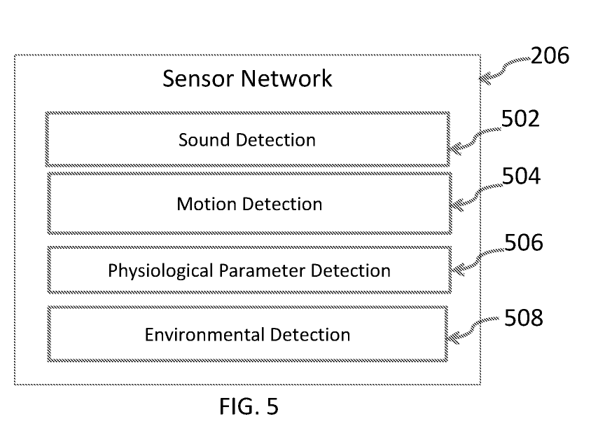

FIG. 5 illustrates a sensor network according to an exemplary embodiment of the present invention.

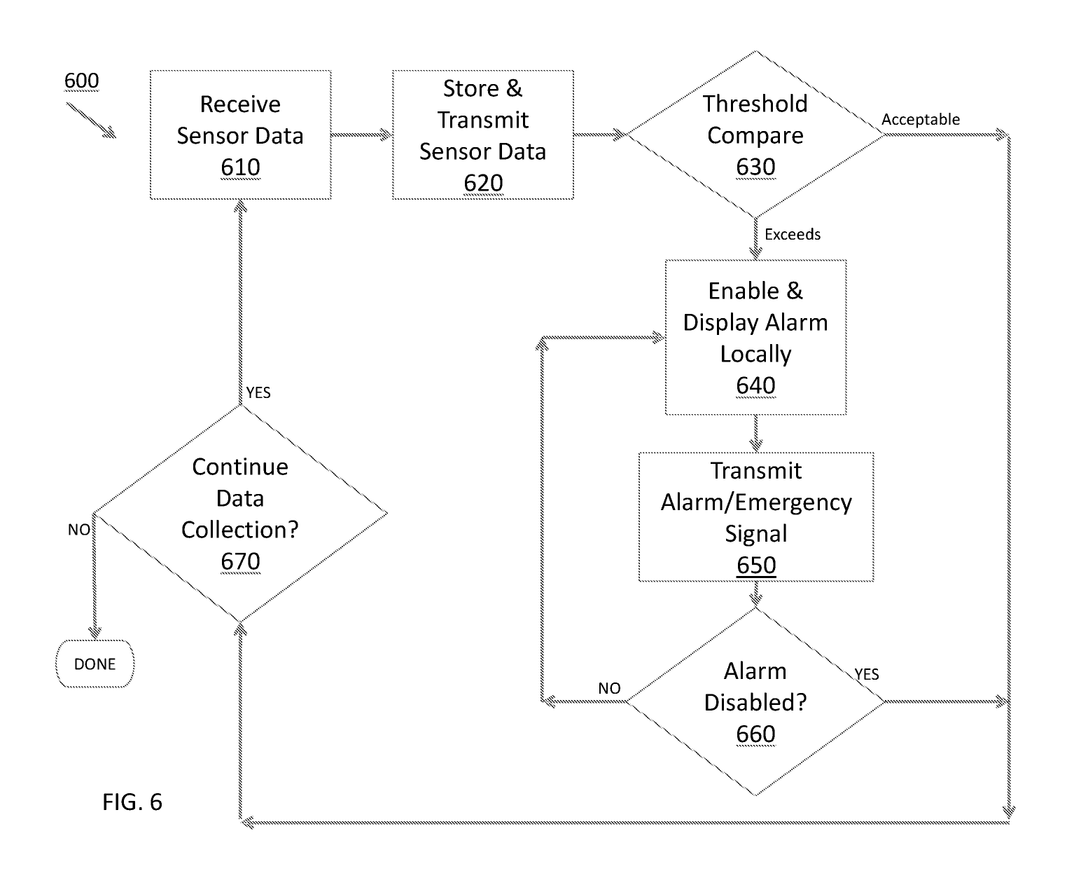

FIG. 6 illustrates control logic according to an exemplary embodiment of the present invention.

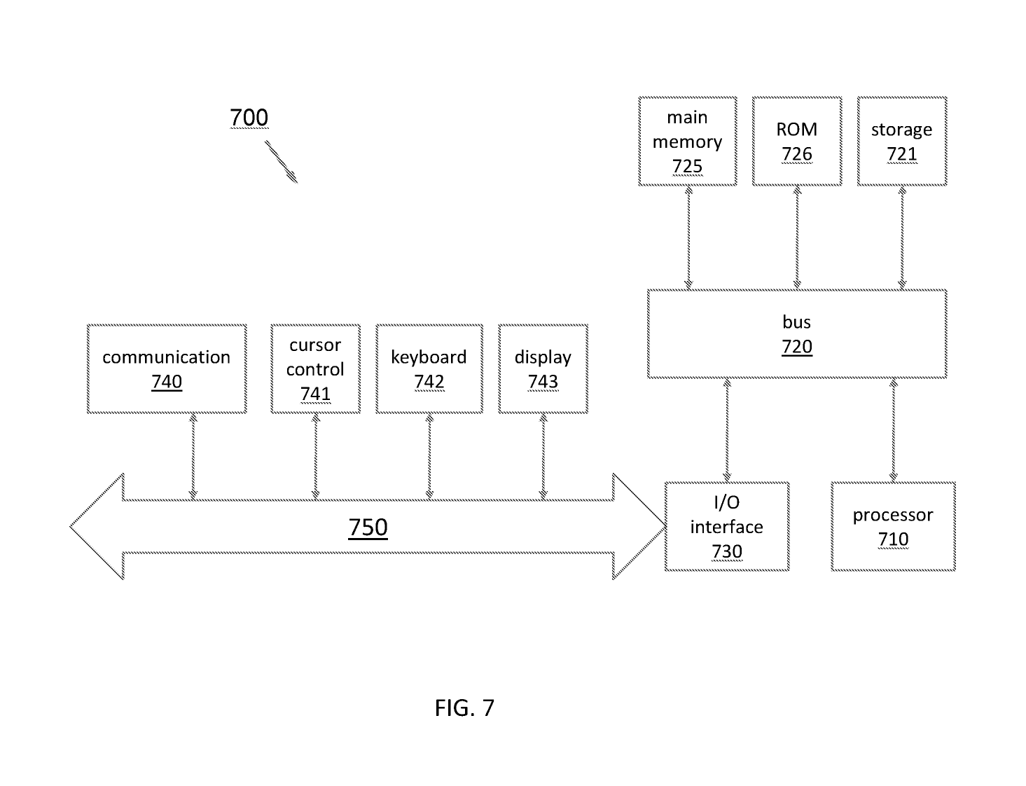

FIG. 7 illustrates an exemplary computer architecture for use with an exemplary embodiment of the present invention.

### DETAILED DESCRIPTION

Reference will now be made in detail to the embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth to provide a thorough understanding of the present invention. However, it will be obvious to one of ordinary skill in the art that the present invention may be practiced without these specific details. In other instances, well-known methods, procedures, components, and circuits have not been described in detail as to not unnecessarily obscure aspects of the present invention.

FIG. 1 illustrates a smart physiological monitoring system 100 according to an exemplary embodiment of the present invention.

In FIG. 1, smart physiological monitoring system 100 comprises a remote server 107 communicably coupled via Internet/communication network 108 and wireless router 109 to monitoring system 1 designated 110. Internet/communication network 108 can be any communication network that allows data to be communicated or transferred from one point to another. Such a network might be wired or wireless as deemed necessary to be consistent with the spirit and scope of the present invention.

Although not shown, monitoring system 110 includes a smart physiological monitoring device for monitoring an infant, child, patient under care, elderly person, or the like.

In FIG. 1, using mobile device 102, user 101 can log into remote server system 107 to access monitoring system 110. Thus, user 101 remotely located from monitoring system 110 can use the remote server system 107 to access various functionalities relating to the smart physiological monitoring device within monitoring system 110.

Similarly, user 103 can also access monitoring system 2 designated 112, which is also communicably coupled to remote server system 107 via Internet/Communication network 108. Specifically, user 103 can employ tablet 104 to access remote server system 107 via Internet/communication network 108.

User 105 can also remotely access monitoring system N designated as 114. Specifically, user 105 can employ laptop computer 106 to access remote server system 107 via Internet/communication network 108.

User 101, User 103, and/or User 105 can then perform functionalities related to each corresponding monitoring system consistent with the spirit and scope of the present invention. Although not shown, further descriptions of various embodiments of the present invention will be described with reference to the following figures.

FIG. 2A illustrates smart physiological monitoring system 110 according to an exemplary embodiment of the present invention.

In FIG. 2A, smart physiological monitoring system 110 shows various exemplary components of monitoring systems 110, 112 and 114 of FIG. 1. Smart physiological monitoring system 110 comprises router 109 communicably coupled to monitoring device 204. Router 109 receives data from Internet/communication network 108 of FIG. 1. Router 109 is a Wi-Fi router capable of receiving data from the Internet and that is compliant with 802.11 standards. Router 109, as mentioned, is capable of receiving data from Internet/communication network 108 and transmitting received data to monitoring device 204.

Such data is transferred wirelessly from router 109 to monitoring device 204. Monitoring device 204 might be located within receiving distance of router 109 such that both components can effectively communicate with each other. Router 202 is also capable of receiving data from monitoring device 204 and transferring that data via Internet/communication network 108 to the remote server system 107 of FIG. 1.

Monitoring device 204 is capable of transmitting data to router 109 and for transfer to remote server system 107 of FIG. 1. Such data might comprise physiological parameters collected by sensors 206 within or in communication with the monitoring device 204.

Monitoring device 204 is also communicably coupled to a sensor network 206. Monitoring device 204 can receive data (e.g., motion, sound, pharmacological vitals, and the like) from the sensor network 206 and push such data via router 109 and Internet/communication network 108 to the remote server system 107 of FIG. 1. The sensor network 206 senses data from a subject of monitoring 202.

Monitoring device 204 is also communicably coupled to mobile device 102 via Bluetooth.

FIGS. 2B and 2C illustrate smart physiological monitoring system 110 according to an exemplary embodiment of the present invention. It will be appreciated that, while monitoring device 204 is depicted as a mobile computing device 204, any computing device with appropriate computing and communication capabilities is applicable as monitoring device 204 without departing from the scope of the present invention. A sensor network 206 is placed near a subject of monitoring 202, and the sensor network 206 delivers data to the monitoring device 204. In one embodiment, the sensor network 206 is internal to the monitoring device 204. In other embodiments, the sensor network 206 is external to the monitoring device 204.

FIG. 3 illustrates remote server system 107 according to an exemplary embodiment of the present invention.

In FIG. 3, remote server system 107 includes web server 302 and application server 304. Web server 302 functions to serve up and host a website (not shown) that can be accessed by user 101, 103, and 105 of FIG. 1. Among other functionalities, users can access this website to determine monitoring subject status information as well as issue corresponding commands to enable or disable monitoring. Web server 302 can be hardware, software or a combination of both.

Application server 304 controls all software applications accessible by users 101, 103, and 105. Users can access monitoring application 306, which comprises one or more software instructions to control and communicate with the monitoring devices within remotely located monitoring systems 110, 112, 114 of FIG. 1. Monitoring application 306 may also include one or more software instructions enabling a user to view monitoring information. Application servers 302 and 304 are communicably coupled to database 308, in which information for all registered users and their monitoring devices are stored.

FIG. 4 illustrates a smart physiological monitoring device 204 according to an exemplary embodiment of the present invention.

In FIG. 4, smart physiological monitoring device 204 includes Wi-Fi circuitry 402, Bluetooth circuitry 404, and RF/IR circuitry 416 that enable wireless communication through their respective protocols.

Wi-Fi circuitry 402 can receive or transmit data to and from router 109 of FIG. 1. Specifically, Wi-Fi circuitry 402 processes remote commands received from users via Internet/Communication network 108 and router 109 before being processed at smart physiological monitoring device 204.

Bluetooth circuitry 404 enables smart physiological monitoring device 204 to establish communication via Bluetooth protocol with any Bluetooth enabled device (e.g. mobile devices 102, 104, 106 of FIG. 1). IR/RF circuitry 416 enables smart physiological monitoring device 204 to establish communication via IR or RF with any IR or RF enabled device. IR circuitry is also used in detection of physiological parameters.

In FIG. 4, smart physiological monitoring device 204 includes control circuitry and digital storage 414. Control circuitry and digital storage 414 provides for processing of instructions to control smart physiological monitoring device 204 and storage of digital information, including collected data, applications and media. Control circuitry and digital storage 414 includes a processor and logic instructions. The logic instructions can be an operating system, for example an Android or Apple operating system that provides for an interface and a rich computing experience for a user. The logic instructions can further include priority logic for deciding between multiple devices attempting to control smart physiological monitoring device 204 at the same time.

Digital storage of smart physiological monitoring device 204 is, in one embodiment, comprised of an internal hard drive, DDR RAM, and a Micro SD card slot for expandable storage. A processor of smart physiological monitoring device 204 is, in one embodiment, a quad core CPU.

In FIG. 4, smart physiological monitoring device 204 further includes a display (not shown) that displays digital information to a user. The display can be a touchscreen. The digital information to be displayed can be retrieved from local storage of smart physiological monitoring device 204 or can be streamed from an external device or server.

In FIG. 4, smart physiological monitoring device 204 further includes a camera and microphone 410. The camera and microphone 410 enable smart physiological monitoring device 204 to capture digital images as well as record video. The camera and microphone 410 further enable smart physiological monitoring device 204 to provide video conferencing functionalities. The video conferencing functionalities enable a user to engage in video conferencing sessions such as those provided by Skype, Google Hangout, and the like.

In FIG. 4, smart physiological monitoring device 204 further includes audio/video rendering capabilities 412.

According to embodiments, captured digital images and digital video (all visible and non-visible wavelengths) are analyzed to detect the presence, the measurement or both of physiological information. The detected physiological information can trigger an alarm if the calculations yield results outside a predetermined threshold for each physiological data set. Physiological information, for example, can include one or more of heart rate, body surface temperature in C or F, respiratory rate in breaths per minute, carbon dioxide output, blood pressure, body movement, sound, oxygen saturation (SPo2) as a numeric value, heart rate in peats per minute, and the like.

According to embodiments, captured digital images and digital video are analyzed at one or a combination of the primary monitoring device and the remote server.

In FIG. 4, smart physiological monitoring device 204 includes one or more sensors 206. Sensor(s) 206 can detect a number of parameters associated with a subject being monitoring and provide the parameter data to control circuitry 414 such that the parameter data can be processed using monitoring logic 406, displayed locally, and transmitted remotely. Sensor(s) 206 can be internal or external to the monitoring device, or a combination of both.

FIG. 5 illustrates a sensor network 206 according to an exemplary embodiment of the present invention.

In FIG. 5, sensor network 206 can include one or more of the following sensors: sound detection 502, motion detection 504, physiological parameter detection 506, environmental detection 508. It will be appreciated that the foregoing is not an exhaustive list of the types of sensors to be included in sensor network 206. It will be appreciated that Infrared circuitry can be used to detect parameters.

In FIG. 5, examples of data to be collected include heart rate, body surface temperature in C or F, respiratory rate in breaths per minute, carbon dioxide output, blood pressure, body movement, sound, oxygen saturation (SPo2) as a numeric value, heart rate in beats per minute, and the like.

FIG. 6 illustrates control logic 600 according to an exemplary embodiment of the present invention.

In FIG. 6, sensor data is received 610. The sensor data is stored locally and transmitted to a remote server 620. The sensor data is then compared to a predefined threshold 630. If the level of the sensor data is acceptable and data collection is to continue 670, more sensor data is received 610. However, if the sensor data is not acceptable (e.g., the data exceeds the threshold) 630, an alarm is locally enabled and displayed 640. An alarm or emergency signal is transmitted 650 to the remote server. This continues until the alarm is disabled 660. Once again, if the data collection is to continue 670, more sensor data is received 610.

FIG. 7 illustrates an exemplary computer architecture 700 for use with an exemplary embodiment of the present invention.

The present invention comprises various computing entities that may have an architecture according to exemplary architecture 700, including monitoring device 204. One embodiment of architecture 700 comprises a system bus 720 for communicating information, and a processor 710 coupled to bus 720 for processing information. Architecture 700 further comprises a random access memory (RAM) or other dynamic storage device 725 (referred to herein as main memory), coupled to bus 720 for storing information and instructions to be executed by processor 710. Main memory 725 also may be used for storing temporary variables or other intermediate information during execution of instructions by processor 710. Architecture 700 may also include a read only memory (ROM) and/or other static storage device 726 coupled to bus 720 for storing static information and instructions used by processor 710.

A data storage device 725 such as a magnetic disk or optical disc and its corresponding drive may also be coupled to architecture 700 for storing information and instructions. Architecture 700 can also be coupled to a second I/O bus 750 via an I/O interface 730. A plurality of I/O devices may be coupled to I/O bus 750, including a display device 743, an input device (e.g., an alphanumeric input device 742 and/or a cursor control device 741).

The communication device 740 allows for access to other computers (e.g., servers or clients) via a network. The communication device 740 may comprise one or more modems, network interface cards, wireless network interfaces or other interface devices, such as those used for coupling to Ethernet, token ring, or other types of networks.

While the above is a complete description of exemplary specific embodiments of the invention, additional embodiments are also possible. Thus, the above description should not be taken as limiting the scope of the invention, which is defined by the appended claims along with their full scope of equivalents.

What is claimed is:

1. A smart physiological monitoring system, comprising:
a monitoring apparatus communicably coupled with an Internet router and a mobile computing device via wireless transmission circuitry, the wireless communication circuitry of the smart physiological monitoring apparatus comprising Wi-Fi communication circuitry and Bluetooth communication circuitry; and
a sensor network, the sensor network comprising at least one physiological parameter sensor.

2. The smart physiological monitoring system of claim 1, wherein the monitoring apparatus comprises a camera and an Infrared sensor.
